(19) United States
(12) Patent Application Publication
(10) Pub. No.: US 2017/0238907 A1
(43) Pub. Date: Aug. 24, 2017

(54) METHODS AND SYSTEMS FOR GENERATING AN ULTRASOUND IMAGE

(71) Applicant: General Electric Company, Schenectady, NY (US)

(72) Inventor: Mohan Krishna Kommu CHS, Bangalore (IN)

(21) Appl. No.: 15/049,702

(22) Filed: Feb. 22, 2016

(51) Int. Cl. A61B 8/08

(52) U.S. Cl. CPC A61B 8/5215; A61B 8/483

(57) ABSTRACT

Systems and methods are provided for generating an ultrasound image. The systems and methods acquire three dimensional (3D) ultrasound data of a volumetric region of interest (ROI) from an ultrasound probe. The systems and methods further identify a select set of the 3D ultrasound data that includes a plurality of anatomical markers. The one or more anatomical markers may correspond to an anatomy of interest within the volumetric ROI. The systems and methods further generate a two dimensional (2D) ultrasound image based on the select set of the 3D ultrasound data, and display the 2D ultrasound image on a display.

### FIELD

Embodiments described herein generally relate to generating one or more ultrasound images of a diagnostic medical imaging system.

### BACKGROUND OF THE INVENTION

Diagnostic medical imaging systems typically include a scan portion and a control portion having a display. For example, ultrasound imaging systems usually include ultrasound scanning devices, such as ultrasound probes having transducers that are connected to an ultrasound system to control the acquisition of ultrasound data by performing various ultrasound scans (e.g., imaging a volume or body). The ultrasound systems are controllable to operate in different modes of operation to perform different scans, for example, to view anatomical structures within the patient.

Conventional ultrasound imaging systems use real-time processing, which requires high performance and high cost processor(s) to display acquired ultrasound images in real-time. While viewing the real-time ultrasound images, users or technicians having high ultrasound expertise will re-position and/or re-orient the ultrasound probe at appropriate scan planes in order to acquire new ultrasound images that include desired anatomical structures. A new method and ultrasound imaging system is desired that does not require expert users and/or high cost processors.

### BRIEF DESCRIPTION OF THE INVENTION

In one embodiment, a method for generating an ultrasound image is provided. The method may include acquiring three dimensional (3D) ultrasound data of a volumetric region of interest (ROI) from an ultrasound probe. The method may further include identifying a select set of the 3D ultrasound data that includes a plurality of anatomical markers. The one or more anatomical markers may correspond to an anatomy of interest within the volumetric ROI. The method may further include generating a two dimensional (2D) ultrasound image based on the select set of the 3D ultrasound data, and displaying the 2D ultrasound image on a display.

In one embodiment, an ultrasound imaging system is provided. The ultrasound imaging system may include an ultrasound probe configured to acquire three dimensional (3D) ultrasound data of a volumetric region of interest (ROI). The ultrasound imaging system may include a display, a memory configured to store programmed instructions, and one or more processors configured to execute the programmed instructions stored on the memory. The one or more processors when executing the programmed instructions perform one or more operations. The one or more operations may include collecting the 3D ultrasound data from the ultrasound probe, and identifying a select set of the 3D ultrasound data that includes a plurality of anatomical markers. The one or more anatomical markers corresponding to an anatomy of interest within the volumetric ROI. The one or more operations may include generating a two dimensional (2D) ultrasound image based on the select set of the 3D ultrasound data, and displaying the 2D ultrasound image on the display.

In one embodiment, a tangible and non-transitory computer readable medium comprising one or more computer software modules is provided. The one or more computer software modules may be configured to direct one or more processors to perform one or more operations. The one or more operations may include acquiring three dimensional (3D) ultrasound data of a volumetric region of interest (ROI) from an ultrasound probe, and identifying a select set of the 3D ultrasound data that includes a plurality of anatomical markers. The one or more anatomical markers may correspond to an anatomy of interest within the volumetric ROI. The one or more operations may further include generating a two dimensional (2D) ultrasound image based on the select set of the 3D ultrasound data, and displaying the 2D ultrasound image on a display.

### BRIEF DESCRIPTION OF THE DRAWINGS

FIG. 1 illustrates a schematic block diagram of an ultrasound imaging system, in accordance with an embodiment.

FIG. 2 is an illustration of a simplified block diagram of a controller circuit of the ultrasound imaging system of FIG. 1, in accordance with an embodiment.

FIG. 3 illustrate a flowchart of a method for generating an ultrasound image, in accordance with an embodiment.

FIG. 4 illustrates a perspective view of a scanned area of a patient, in accordance with an embodiment.

FIGS. 5A-B illustrate frames of three dimensional ultrasound data, in accordance with an embodiment.

FIG. 6 illustrates a flowchart of a method for identifying a select set of three dimensional ultrasound data that includes a plurality of anatomical markers, in accordance with an embodiment.

FIG. 7 illustrates the frames of the three dimensional ultrasound data of FIG. 5B with orthogonal planes.

FIG. 8 illustrates a defined two dimensional plane within three dimensional ultrasound data based on one or more anatomical markers, in accordance with an embodiment.

FIG. 9 illustrates a graphical user interface of a plurality of two dimensional ultrasound images, in accordance with an embodiment.

FIG. 10 illustrates a graphical user interface of a two dimensional ultrasound image, in accordance with an embodiment.

FIG. 11 illustrates a 3D capable miniaturized ultrasound system having a probe that may be configured to acquire 3D ultrasonic data or multi-plane ultrasonic data.

FIG. 12 illustrates a hand carried or pocket-sized ultrasound imaging system wherein the display and user interface form a single unit.

FIG. 13 illustrates an ultrasound imaging system provided on a movable base.

### DETAILED DESCRIPTION OF THE INVENTION

The following detailed description of certain embodiments will be better understood when read in conjunction with the appended drawings. To the extent that the figures illustrate diagrams of the functional modules of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments provide systems and methods that allow a user (e.g., clinician, technician) to complete an anatomical examination such as an obstetrics (OB) examination without having to view real-time or live ultrasound images during the examination. In operation, the user scans a region of interest (ROI) of the patient, such as the abdomen, using an ultrasound probe. For example, the clinician may scan the ROI by repeatingly moving the ultrasound probe from left to right starting from the top left side of the ROI moving downwards.

Various embodiments capture ultrasound frames of the entire abdomen as beam formed data (before scan converting the image into a fan beam), such as 3D ultrasound data that includes vector data stored in a memory. In operation, various embodiments may include one or more processors that execute an imaging algorithm stored on memory. When executing the imaging algorithm, the one or more processors analyze the 3D ultrasound data, and identify frames containing anatomical markers. The anatomical markers may be a fetal head, an abdomen, a femur, a position of fetal head, and/or the like. To identify the anatomical markers, the imaging algorithm may match patterns of the 3D ultrasound data that correspond to the anatomical markers. For example to identify an anatomical marker of a fetal head, the one or more processors executing the imaging algorithm may identify an elliptical outer line and mid line pattern.

In operation, when select sets of the 3D ultrasound data are identified having the anatomical markers, various embodiments generate 2D ultrasound images from the select sets of the 3D ultrasound data. The 2D ultrasound images may be displayed on a display. Optionally, a plurality of the 2D ultrasound images may be viewed concurrently on the display, for example in a grid or matrix view. The plurality of 2D ultrasound images corresponding to different anatomies of interest. Additionally or alternatively, various embodiments may generate additional 2D ultrasound images corresponding to the same anatomy of interest based on different select sets of the 3D ultrasound data, which allow the user to have a choice to select one of the 2D ultrasound images. Optionally, the user may select one or more of the 2D ultrasound images on the display to perform one or more diagnostic measurements.

A technical effect of at least one embodiment described herein allows a user to sweep a ROI to acquire 2D ultrasound images of an anatomy of interest rather than attempting to position an ultrasound probe at a select scan plane. A technical effect of at least one embodiment described herein increases processing efficiency by only generating 2D ultrasound images based on frames that include the anatomy of interest.

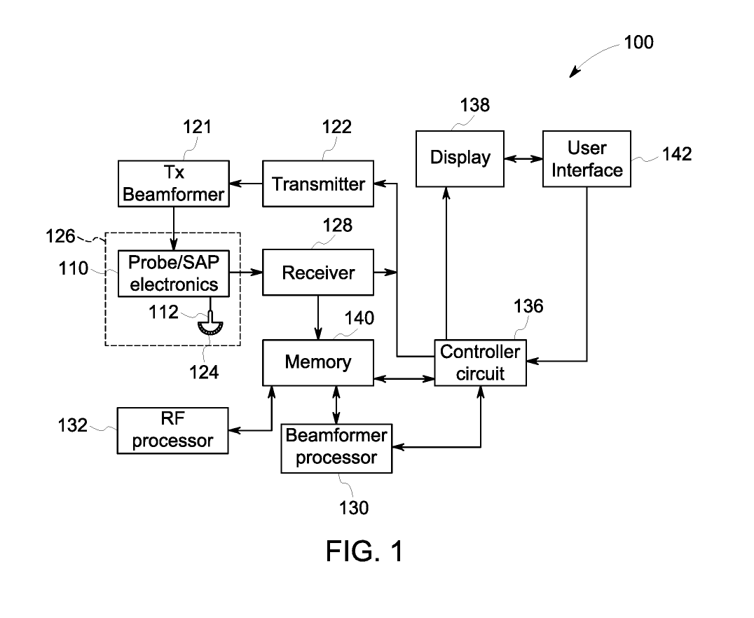

FIG. 1 is a schematic diagram of a diagnostic medical imaging system, specifically, an ultrasound imaging system 100. The ultrasound imaging system 100 includes an ultrasound probe 126 having a transmitter 122 and probe/SAP electronics 110. The ultrasound probe 126 may be configured to acquire ultrasound data or information from a region of interest (e.g., organ, blood vessel, heart) of the patient. The ultrasound probe 126 is communicatively coupled to the controller circuit 136 via the transmitter 122. The transmitter 122 transmits a signal to a transmit beamformer 121 based on acquisition settings received by the user. The signal transmitted by the transmitter 122 in turn drives the transducer elements 124 within the transducer array 112. The transducer elements 124 emit pulsed ultrasonic signals into a patient (e.g., a body). A variety of a geometries and configurations may be used for the array 112. Further, the array 112 of transducer elements 124 may be provided as part of, for example, different types of ultrasound probes. Optionally, the ultrasound probe 126 may include one or more tactile buttons (not shown). For example, a pressure sensitive tactile button may be positioned adjacent to the transducer array 122 of the ultrasound probe 126. In operation, when the transducer array 112 and/or generally the ultrasound probe 126 is in contact with the patient during acquisition of ultrasound data the pressure sensitive tactile button may be activated.

The acquisition settings may define an amplitude, pulse width, frequency, and/or the like of the ultrasonic pulses emitted by the transducer elements 124. The acquisition settings may be adjusted by the user by selecting a gain setting, power, time gain compensation (TGC), resolution, and/or the like from the user interface 142.

The transducer elements 124, for example piezoelectric crystals, emit pulsed ultrasonic signals into a body (e.g., patient) or volume corresponding to the acquisition settings along one or more scan planes. The ultrasonic signals may include, for example, one or more reference pulses, one or more pushing pulses (e.g., shear-waves), and/or one or more pulsed wave Doppler pulses. At least a portion of the pulsed ultrasonic signals back-scatter from a region of interest (ROI) (e.g., abdomen, chest, torso, and/or the like) to produce echoes. The echoes are delayed in time and/or frequency according to a depth or movement, and are received by the transducer elements 124 within the transducer array 112. The ultrasonic signals may be used for imaging, for generating and/or tracking shear-waves, for measuring changes in position or velocity within the ROI, differences in compression displacement of the tissue (e.g., strain), and/or for therapy, among other uses.

The transducer array 112 may have a variety of array geometries and configurations for the transducer elements 124 which may be provided as part of, for example, different types of ultrasound probes 126. The probe/SAP electronics 110 may be used to control the switching of the transducer elements 124. The probe/SAP electronics 110 may also be used to group the transducer elements 124 into one or more sub-apertures.

The transducer elements 124 convert the received echo signals into electrical signals which may be received by a receiver 128. The receiver 128 may include one or more amplifiers, an analog to digital converter (ADC), and/or the like. The receiver 128 may be configured to amplify the received echo signals after proper gain compensation and convert these received analog signals from each transducer element 124 to digitized signals sampled uniformly in time. The digitized signals representing the received echoes are stored on memory 140, temporarily. The digitized signals correspond to the backscattered waves receives by each transducer element 124 at various times. After digitization, the signals still may preserve the amplitude, frequency, phase information of the backscatter waves.

Optionally, the controller circuit 136 may retrieve the digitized signals stored in the memory 140 to prepare for the beamformer processor 130. For example, the controller circuit 136 may convert the digitized signals to baseband signals or compressing the digitized signals.

The beamformer processor 130 may include one or more processors. Optionally, the beamformer processor 130 may include a central controller circuit (CPU), one or more microprocessors, or any other electronic component capable of processing inputted data according to specific logical instructions. Additionally or alternatively, the beamformer processor 130 may execute instructions stored on a tangible and non-transitory computer readable medium (e.g., the memory 140) for beamforming calculations using any suitable beamforming method such as adaptive beamforming, synthetic transmit focus, aberration correction, synthetic aperture, clutter reduction and/or adaptive noise control, and/or the like.

The beamformer processor 130 may further perform filtering and decimation, such that only the digitized signals corresponding to relevant signal bandwidth is used, prior to beamforming of the digitized data. For example, the beamformer processor 130 may form packets of the digitized data based on scanning parameters corresponding to focal zones, expanding aperture, imaging mode (B-mode, color flow), and/or the like. The scanning parameters may define channels and time slots of the digitized data that may be beamformed, with the remaining channels or time slots of digitized data that may not be communicated for processing (e.g., discarded).

The beamformer processor 130 performs beamforming on the digitized signals and outputs a radio frequency (RF) signal. The RF signal is then provided to an RF processor 132 that processes the RF signal. The RF processor 132 may generate different ultrasound image data types, e.g. B-mode, for multiple scan planes or different scanning patterns. The RF processor 132 gathers the information (e.g. I/Q, B-mode) related to multiple data slices and stores the data information, which may include time stamp and orientation/rotation information, in the memory 140.

Alternatively, the RF processor 132 may include a complex demodulator (not shown) that demodulates the RF signal to form IQ data pairs representative of the echo signals. The RF or IQ signal data may then be provided directly to the memory 140 for storage (e.g., temporary storage). Optionally, the output of the beamformer processor 130 may be passed directly to the controller circuit 136.

The controller circuit 136 may be configured to process the acquired ultrasound data (e.g., RF signal data or IQ data pairs) and identify select sets and/or portion of the ultrasound data that include a plurality of anatomical markers within the ROI that corresponding to an anatomy of interest. The controller circuit 136 may further prepare frames of the select sets of the ultrasound data to generate ultrasound images for display on the display 138. The controller circuit 136 may include one or more processors. Optionally, the controller circuit 136 may include a central controller circuit (CPU), one or more microprocessors, a graphics controller circuit (GPU), or any other electronic component capable of processing inputted data according to specific logical instructions. Having the controller circuit 136 that includes a GPU may be advantageous for computation-intensive operations, such as volume-rendering. Additionally or alternatively, the controller circuit 136 may execute instructions stored on a tangible and non-transitory computer readable medium (e.g., the memory 140) to perform one or more operations as described herein.

The controller circuit 136 may be configured to perform one or more processing operations to identify portions of the ultrasound data that include a plurality of anatomical markers corresponding to an anatomy of interest within the ROI, adjust or define the ultrasonic pulses emitted from the transducer elements 124 based on the anatomy of interest and/or scan being performed by the user, adjust one or more image display settings of components (e.g., ultrasound images, interface components, positioning regions of interest) displayed on the display 138, and other operations as described herein. Acquired ultrasound data may be processed by the controller circuit 136 during a scanning or therapy session as the echo signals are received.

The memory 140 may be used for storing ultrasound data such as vector data, processed frames of acquired ultrasound data that are not scheduled to be displayed immediately or to store post-processed images, firmware or software corresponding to, for example, a graphical user interface, one or more default image display settings, programmed instructions (e.g., for the controller circuit 136, the beamformer processor 130, the RF processor 132), and/or the like. The memory 140 may be a tangible and non-transitory computer readable medium such as flash memory, RAM, ROM, EEPROM, and/or the like.

In operation, the ultrasound data may include and/or correspond to three dimensional (3D) ultrasound data. The memory 140 may store the 3D ultrasound data, where the 3D ultrasound data or select sets of the 3D ultrasound data are accessed by the controller circuit 136 to generate 2D ultrasound images. For example, a 3D ultrasound data may be mapped into the corresponding memory 140, as well as one or more reference planes. The processing of the 3D ultrasound data may be based in part on user inputs, for example, user selections received at the user interface 142.

The controller circuit 136 is operably coupled to a display 138 and a user interface 142. The display 138 may include one or more liquid crystal displays (e.g., light emitting diode (LED) backlight), organic light emitting diode (OLED) displays, plasma displays, CRT displays, and/or the like. The display 138 may display patient information, ultrasound images and/or videos, components of a display interface, one or more 2D, 3D, or 4D ultrasound image data sets from ultrasound data stored in the memory 140, measurements, diagnosis, treatment information, and/or the like received by the display 138 from the controller circuit 136.

The user interface 142 controls operations of the controller circuit 136 and is configured to receive inputs from the user. The user interface 142 may include a keyboard, a mouse, a touchpad, one or more physical buttons, and/or the like. Optionally, the display 138 may be a touch screen display, which includes at least a portion of the user interface 142.

For example, a portion of the user interface 142 may correspond to a graphical user interface (GUI) generated by the controller circuit 136, which is shown on the display. The GUI may include one or more interface components that may be selected, manipulated, and/or activated by the user operating the user interface 142 (e.g., touch screen, keyboard, mouse). The interface components may be presented in varying shapes and colors, such as a graphical or selectable icon, a slide bar, a cursor, and/or the like. Optionally, one or more interface components may include text or symbols, such as a drop-down menu, a toolbar, a menu bar, a title bar, a window (e.g., a pop-up window) and/or the like. Additionally or alternatively, one or more interface components may indicate areas within the GUI for entering or editing information (e.g., patient information, user information, diagnostic information), such as a text box, a text field, and/or the like.

In various embodiments, the interface components may perform various functions when selected, such as measurement functions, editing functions, database access/search functions, diagnostic functions, controlling acquisition settings, and/or system settings for the ultrasound imaging system 100 and performed by the controller circuit 136.

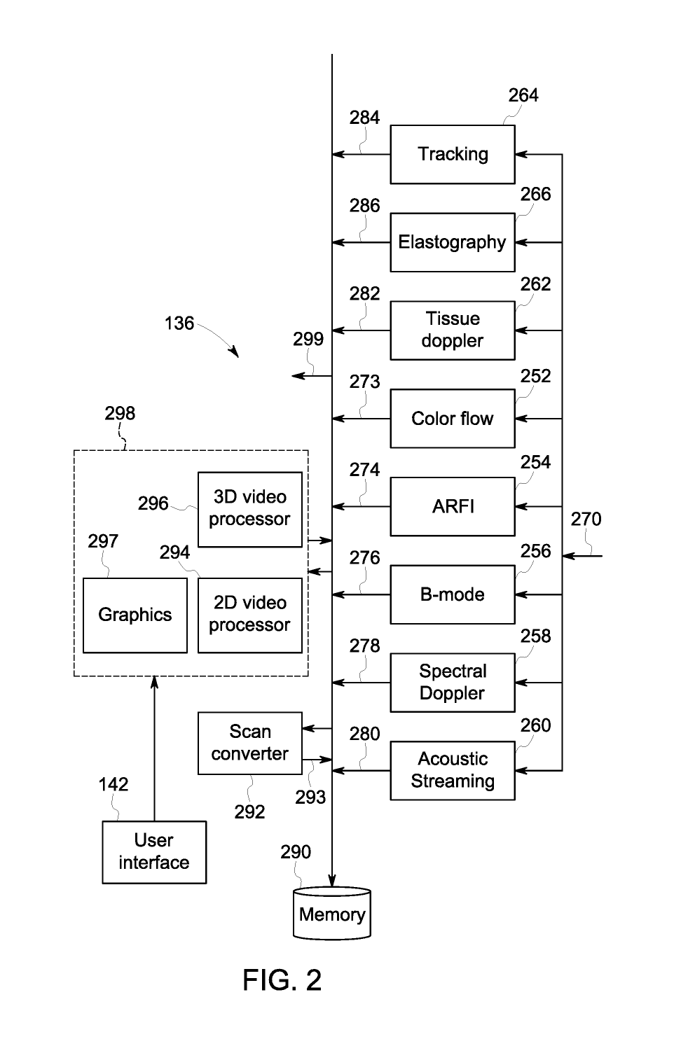

FIG. 2 is an exemplary block diagram of the controller circuit 136. The controller circuit 136 is illustrated in FIG. 2 conceptually as a collection of circuits and/or software modules, but may be implemented utilizing any combination of dedicated hardware boards, DSPs, one or more processors, FPGAs, ASICs, a tangible and non-transitory computer readable medium configured to direct one or more processors, and/or the like.

The circuits 252-266 perform mid-processor operations representing one or more operations or modalities of the ultrasound imaging system 100. The controller circuit 136 may receive ultrasound data 270 (e.g., 3D ultrasound data) in one of several forms. In the embodiment of FIG. 1, the received ultrasound data 270 constitutes IQ data pairs representing the real and imaginary components associated with each data sample of the digitized signals. The IQ data pairs are provided to one or more circuits, for example, a color-flow circuit 252, an acoustic radiation force imaging (ARFI) circuit 254, a B-mode circuit 256, a spectral Doppler circuit 258, an acoustic streaming circuit 260, a tissue Doppler circuit 262, a tracking circuit 264, and an electrography circuit 266. Other circuits may be included, such as an M-mode circuit, power Doppler circuit, among others. However, embodiments described herein are not limited to processing IQ data pairs. For example, processing may be done with RF data and/or using other methods. Furthermore, data may be processed through multiple circuits.

Each of circuits 252-266 is configured to process the IQ data pairs in a corresponding manner to generate, respectively, color flow data 273, ARFI data 274, B-mode data 276, spectral Doppler data 278, acoustic streaming data 280, tissue Doppler data 282, tracking data 284, electrography data 286 (e.g., strain data, shear-wave data), among others, all of which may be stored in a memory 290 (or the memory 140 shown in FIG. 1) temporarily before subsequent processing. The data 273-286 may be stored, for example, as sets of vector data values, where each set defines an individual ultrasound image frame. The vector data values are generally organized based on the polar coordinate system.

In various embodiments the controller circuit 136 may analyze the 3D ultrasound data that include vector data values (e.g., corresponding to the ultrasound data) stored in the memory 140, 290, and to identify portions or sets of the 3D ultrasound data that includes a plurality of anatomical markers. For example, the controller circuit 136 may execute a pattern recognition algorithm stored in the memory 140. When executing the pattern recognition algorithm, the controller circuit 136 may identify intensity changes and/or gradients of the vector data values to identify shapes, contours, and/or the like corresponding to anatomical markers. The locations of the anatomical markers may form one or more patterns that are identified as one or more anatomical structures by the controller circuit 136. For example, the controller circuit 136 may compare portions of each pattern with a plurality of patterns stored in the memory 140, 290 each with a corresponding anatomical structure.

The controller circuit 136 may identify a select set of the vector data values that includes the anatomical markers. For example, when the controller circuit 136 identifies the anatomical structures based on the patterns formed by the anatomical markers, the controller circuit 136 may select a portion of the vector data values corresponding to the anatomical structure. In operation, the select set or portion of the vector data may form a 2D plane of the anatomical structure that includes the anatomical markers. Optionally, the controller circuit 136 may identify multiple 2D planes that include the anatomical structure. For example, the controller circuit 136 may identify multiple adjacent 2D planes that include the anatomical structure, each 2D plane including different vector values of the 3D ultrasound data.

A scan converter circuit 292 accesses and obtains from the memory 290 the select set(s) of the vector data values associated with a 2D ultrasound image frame and converts the set of vector data values to Cartesian coordinates to generate one or more 2D ultrasound image frames 293 formatted for display. The ultrasound image frames 293 generated by the scan converter circuit 292 may be provided back to the memory 290 for subsequent processing or may be provided to the memory 140. Once the scan converter circuit 292 generates the ultrasound image frames 293 associated with the data, the image frames may be stored in the memory 290 or communicated over a bus 299 to a database (not shown), the memory 140, and/or to other processors (not shown).

The display circuit 298 accesses and obtains one or more of the image frames from the memory 290 and/or the memory 140 over the bus 299 to display the images onto the display 138. The display circuit 298 receives user input from the user interface 142 selecting one or image frames to be displayed that are stored on memory (e.g., the memory 290) and/or selecting a display layout or configuration for the image frames.

The display circuit 298 may include a 2D video processor circuit 294. The 2D video processor circuit 294 may be used to combine one or more of the frames generated from the different types of ultrasound information. Successive frames of images may be stored as a cine loop (4D images) in the memory 290 or memory 140. The cine loop represents a first in, first out circular image buffer to capture image data that is displayed in real-time to the user. The user may freeze the cine loop by entering a freeze command at the user interface 142.

The display circuit 298 may include a 3D processor circuit 296. The 3D processor circuit 296 may access the memory 290 to obtain spatially consecutive groups of ultrasound image frames and to generate three-dimensional image representations thereof, such as through volume rendering or surface rendering algorithms as are known. The three-dimensional images may be generated utilizing various imaging techniques, such as ray-casting, maximum intensity pixel or voxel projection and the like.

The display circuit 298 may include a graphic circuit 297. The graphic circuit 297 may access the memory 290 to obtain groups of ultrasound image frames that have been stored or that are currently being acquired. The graphic circuit 297 may generate ultrasound images that include the anatomical structures within the ROI.

Additionally or alternatively, during acquisition of the ultrasound data, the graphic circuit 297 may generate a graphical representation, which is displayed on the display 138. The graphical representation may be used to indicate the progress of the therapy or scan performed by the ultrasound imaging system 100. The graphical representations may be generated using a saved graphical image or drawing (e.g., computer graphic generated drawing).

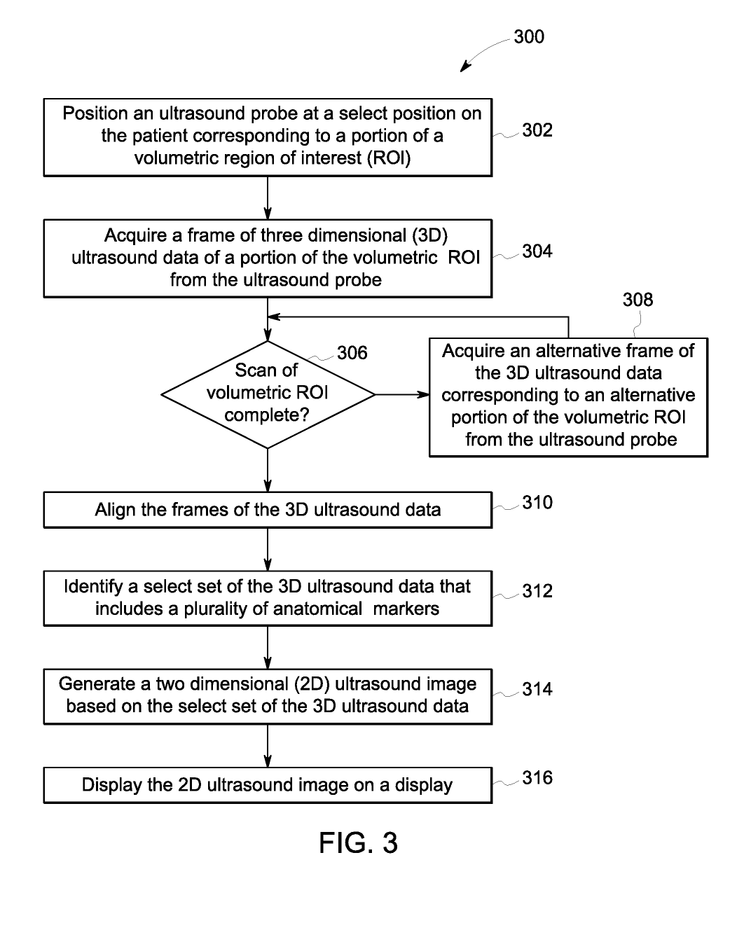

In connection with FIG. 3, the user may select an interface component corresponding to a select scan, which generates one or more 2D ultrasound images of a select anatomical structure using the user interface 142. For example, the select scan may correspond to an OB examination, abdominal scans, urological scans, gastroenterology scans, and/or the like. The 2D ultrasound image may be a B-mode ultrasound image based on the vector data values corresponding to the B-mode data 276. When the interface component is selected, the controller circuit 136 may perform one or more of the operations described in connection with method 300.

FIG. 3 illustrate a flowchart of a method 300 for generating an ultrasound image, in accordance with various embodiments described herein. The method 300, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 300 may be used as one or more algorithms to direct hardware to perform one or more operations described herein. It may be noted, other methods may be used, in accordance with embodiments herein.

One or more methods may (i) acquire three dimensional (3D) ultrasound data of a volumetric region of interest (ROD from an ultrasound probe, (ii) identify a select set of the 3D ultrasound data that includes a plurality of anatomical markers, (iii) generate a two dimensional (2D) ultrasound image based on the select set of the 3D ultrasound data, and (iv) display the 2D ultrasound image on a display.

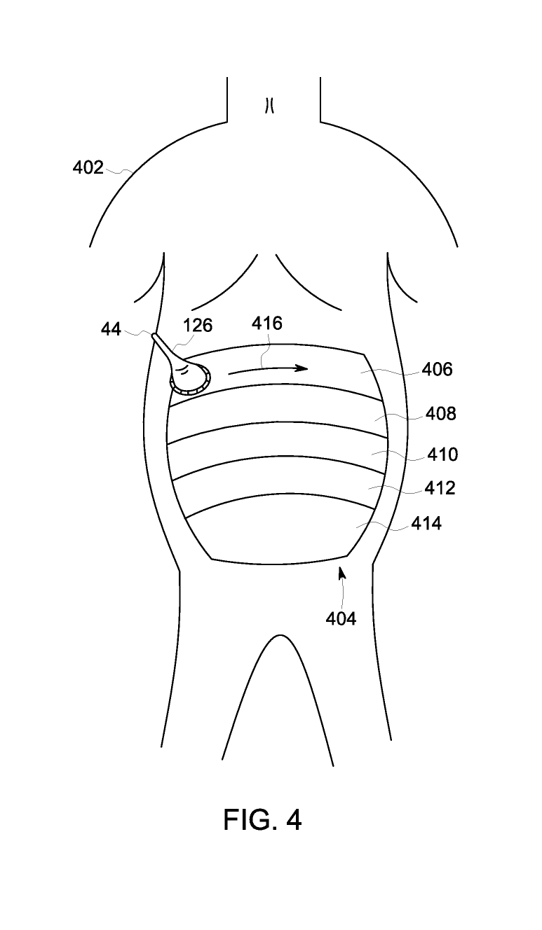

Beginning at 302, the ultrasound probe 126 may be positioned at a select position on the patient corresponding to a portion of a volumetric ROI. FIG. 4 illustrates a perspective view of a scanning area 404 of a patient 402, in accordance with an embodiment. The scanning area 404 may correspond to a position of the volumetric ROI within the patient 402, which includes one or more anatomies of interest. For example, the scanning area 404 illustrated in FIG. 4 may be based on a volumetric ROI corresponding to fetal tissue within the abdomen of the patient 402. The scanning area 404 is shown subdivided into multiple sweeps 406-414. Each sweep 406-414 may correspond to a portion of the scanning area 404 that may be acquired by the ultrasound probe 126 when moving in a direction of an arrow 416. In operation, the user may traverse the ultrasound probe 126 along each sweep 406-414 to acquire the 3D ultrasound data for the scanning area 404. For example, the user may position the ultrasound probe 126 at a starting or select position approximate to an edge of the sweep 406 within the scanning area 404. During the scan, the user may move the ultrasound probe 126 in the direction of the arrow 416 stopping at an opposing side of the sweep 406 with respect to the starting or select position, and repeating the scan at an alternative sweep, for example by repositioning the ultrasound probe 126 at a starting position at the sweep 408.

The anatomy of interest of the scanning area 404 may include an internal organ, fetal head, fetal abdomen, femur, and/or the like. The controller circuit 136 may identify the anatomy of interest based on a predetermined scan selected from a plurality of candidate predetermined scans stored in the memory 140. For example, each predetermined scan (e.g., OB examination, abdominal examination) may include one or more anatomies of interest. The user may select the predetermined scan by selecting one or more interface components shown on the display 138 (e.g., drop down menus) and/or by selecting one or more hotkeys on the user interface 142.

In operation, the controller circuit 136 may automatically adjust the acquisition settings of the ultrasound probe 126 based on the predetermined scan. For example, the predetermined scan (e.g., OB examination) may include an anatomy of interest based on a volumetric ROI of a fetus within the patient 402. The controller circuit 136 may adjust the acquisition settings, such as the amplitude, pulse width, frequency and/or the like of the ultrasound pulses emitted by the transducer elements 124 of the ultrasound probe 126 based on a depth and/or position of the fetus within the patient 402.

At 304, the controller circuit 136 may acquire a frame of the 3D ultrasound data of a portion of the volumetric ROI from the ultrasound probe 126. A frame of the 3D ultrasound data may be based on the sweep 406-414. In operation, the area of the volumetric ROI represented by the frame of the 3D ultrasound data may be defined by the corresponding sweep 406-414 scanned by the ultrasound probe 126. For example, as the ultrasound probe 126 moves and/or traverses along the sweep 406 the transducer elements 124 may transmit ultrasonic pulses. It may be noted that a portion the 3D ultrasound data in frames acquired at adjacent sweeps may be the same, such as at and/or approximate to the edges of the frames. For example, a first portion of the 3D ultrasound data within a frame acquired along the sweep 408 may be the same as a portion of the 3D ultrasound data within a frame acquired along the sweep 406. In another example, a second portion of the 3D ultrasound data within the frame acquired along the sweep 408 may be the same as apportion of the 3D ultrasound data within a frame acquired along the sweep 410.

Optionally, the controller circuit 136 may instruct the ultrasound probe 126 to begin transmitting ultrasonic pulses based on a received input from the user interface 142 and/or activation of a tactile button on the ultrasound probe 126. For example, the tactile button may be a pressure sensitive button that is activated when the transducer array 112 and/or generally the ultrasound probe 126 is in contact with and/or proximate (e.g., within a predetermined distance) to the patient 404 during a scan (e.g., traversing within the sweep 406-414). Additionally, the pressure sensitive button may be deactivated when the ultrasound probe 126 is not in contact with and/or outside a predetermined distance (e.g., 5 cm, 10 cm) from the patient 404. A status (e.g., activated, deactivated) of the pressure sensitive button may be received by the controller circuit 136. When the pressure sensitive button is activated, the controller circuit 136 may determine that the patient 404 is being scanned, and instructs the ultrasound probe 126 to transmit the ultrasonic pulses.

At least a portion of the ultrasound pulses are backscattered by the tissue of the volumetric ROI positioned within the sweep 406, and are received by the receiver 128. The receiver 128 converts the received echo signals into digitized signals. The digitized signals, as described herein, are beamformed by the beamformer processor 130 and formed into IQ data pairs representative of the echo signals by the RF processor 132, and are received as the ultrasound data 270 (e.g., the 3D ultrasound data) by the controller circuit 136. The ultrasound data 270, which corresponds to the 3D ultrasound data, may be processed by the B-mode circuit 256 or generally the controller circuit 136. The B-mode circuit 256 may process the IQ data pairs to generate B-mode data 276, for example, sets of vector data values forming a frame of the 3D ultrasound data stored in the memory 290 or the memory 140.

Optionally as the frame of the 3D ultrasound data is being acquired, the display 138 may display a graphical representation, such as a progress bar. The graphical representation may include numerical information (e.g., percentage), a color code corresponding to a proportion of the scan completed, and/or the like. For example, the graphical representation may be a visualization of a progression of the scan and/or status of the acquisition of the 3D ultrasound data of the volumetric ROI.

Additionally or alternatively, as the frame of the 3D ultrasound data is being acquired the display 138 may not display a real-time ultrasound image and/or any ultrasound image from simultaneously acquired 3D ultrasound data, from 3D ultrasound data acquired during the same predetermined scan (e.g., during the scanning session), while concurrently acquiring 3D ultrasound data, from 3D ultrasound data acquired after a processing delay of the controller circuit 136, and/or the like.

At 306 the controller circuit 136 may determine whether the scan of the volumetric ROI is completed. If the scan of the volumetric ROI is not complete, the controller circuit 136 may acquire, at 308, an alternative frame of the 3D ultrasound data corresponding to an alternative portion of the volumetric ROI from the ultrasound probe. For example, the controller circuit 136 may determine when all of the 3D ultrasound data corresponding to the frame is acquired based on the status of the one or more tactile buttons (e.g., the pressure sensitive button).

In operation, when the ultrasound probe 126 is moved and/or positioned to an alternative sweep 406-414 for a subsequent frame, such as from the sweep 406 to the sweep 408, the controller circuit 136 may detect changes in an activation state of the tactile button. For example, when the ultrasound probe 126 is being moved and/or repositioned from the sweep 406 to the sweep 408 the controller circuit 136 may detect deactivation of the tactile button. When the controller circuit 136 detects the deactivation of the tactile button, the controller circuit 136 may determine that the acquisition of the 3D ultrasound data for the frame is complete. Additionally or alternatively, the controller circuit 136 may determine that the acquisition of the frame is complete based on signal received from the user interface 142.

The controller circuit 136 may determine whether the scan is completed based on a length of the deactivation of the one or more tactile buttons (e.g., pressure sensitive button) of the ultrasound probe 126. In operation, the controller circuit 136 may monitor a length of time corresponding to deactivation of the one or more tactile buttons. The controller circuit 136 may compare the length of time with a predetermined time period, such as one minute, two minutes, and/or the like. For example, after the ultrasound probe 126 acquires a frame of the 3D ultrasound data corresponding to the sweep 414 the ultrasound probe 126 may no longer be in contact with and/or proximate to the patient 404, such as docked, deactivating the one or more tactile buttons. When the controller circuit 136 determines that the one or more tactile buttons have been deactivated for longer than the predetermined time period, the controller circuit 136 may determine that the scan of the volumetric ROI is complete.

Additionally or alternatively, the controller circuit 136 may determine that an alternative frame is being acquired when the activation of the one or more tactile buttons (e.g., the pressure sensitive button) are detected prior to the predetermined time period. For example, when the ultrasound probe 126 is moved from the sweep 406 and positioned at a select position of the sweep 408 in contact and/or proximate to the patient 404, the one or more tactile buttons may be activated within the predetermined time period.

Additionally or alternatively, the controller circuit 136 may receive a completion signal from the user interface 142 and/or a tactile button on the ultrasound probe 126 when the acquisition of the 3D ultrasound data is complete. For example, the controller circuit 136 may receive a signal from the user interface 142 corresponding to completion of the scan.

At 310, the controller circuit 136 may align a series of frames of the 3D ultrasound data. In operation, the controller circuit 136 may align edges of successively acquired frames of the 3D ultrasound data together to represent the volumetric ROI. For example, the edges may correspond to 3D ultrasound data of successive and/or adjacent frames with similar and/or the same 3D ultrasound data. The controller circuit 136 may stitch and/or align the portions of the 3D ultrasound data that is duplicated or the same in the adjacent frame.

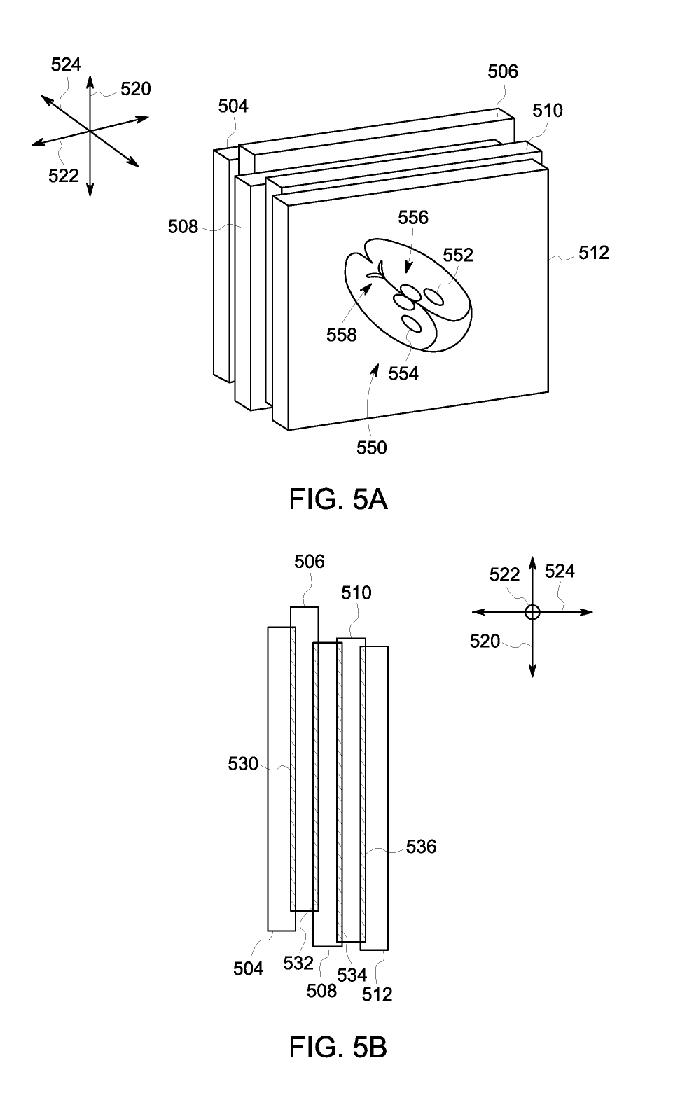

FIGS. 5A-B illustrate frames 504-512 of 3D ultrasound data, in accordance with an embodiment. FIG. 5A illustrates a perspective view of the frames 504-512 of the 3D ultrasound data, and FIG. 5B illustrates a side view of the frames 504-512. Each frame 504-512 of the 3D ultrasound data may correspond to one of the sweeps 406-414, respectively, traversed by the ultrasound probe 126 when acquiring the 3D ultrasound data. The controller circuit 136 may register the series of frames of the 3D ultrasound data by stitching portions (e.g., 530-536) of the 3D ultrasound data duplicated in adjacent frames together. For example, the frames 504 and the frame 506 corresponding to the sweeps 406 and 408, respectively, may each include a portion 530 of duplicated 3D ultrasound data. The controller circuit 136 may adjust a position of the frame 506 along axes 520-524 relative to the frame 504 to align the portion 530 of the frame 504 with the portion 530 of the frame 506. The controller circuit 136 may repeat the alignment of each successive frame 508-512 with respect to the proceeding frame 506-510, respectively.

At 312, the controller circuit 136 may identify a select set of the 3D ultrasound data that includes a plurality of anatomical markers. Optionally, the select set of the 3D ultrasound data may be identified when the completion signal is received by the controller circuit 136 from the user interface 142. In connection with FIG. 5A, the one or more anatomical markers 552-558 may correspond to an anatomy of interest 550 within the volumetric ROI. For example, the anatomical markers 552 and 554 may represent atriums, the anatomical marker 556 may represent the thalamus, and the anatomical marker 558 may represent the cavum septum pellucidum (CSP). Positions of the anatomical markers 552-558 with respect to each other form a pattern representing the anatomy of interest 550. For example, the anatomy of interest 550 illustrated in FIG. 5A may represent a fetal head. Additionally or alternatively, the anatomy of interest may be a fetal femur, fetal abdomen, internal organ, and/or the like. It may be noted, in at least one embodiment the controller circuit 136 may identify a plurality of sets of the 3D ultrasound data each corresponding to a different anatomy of interest within the volumetric ROI.

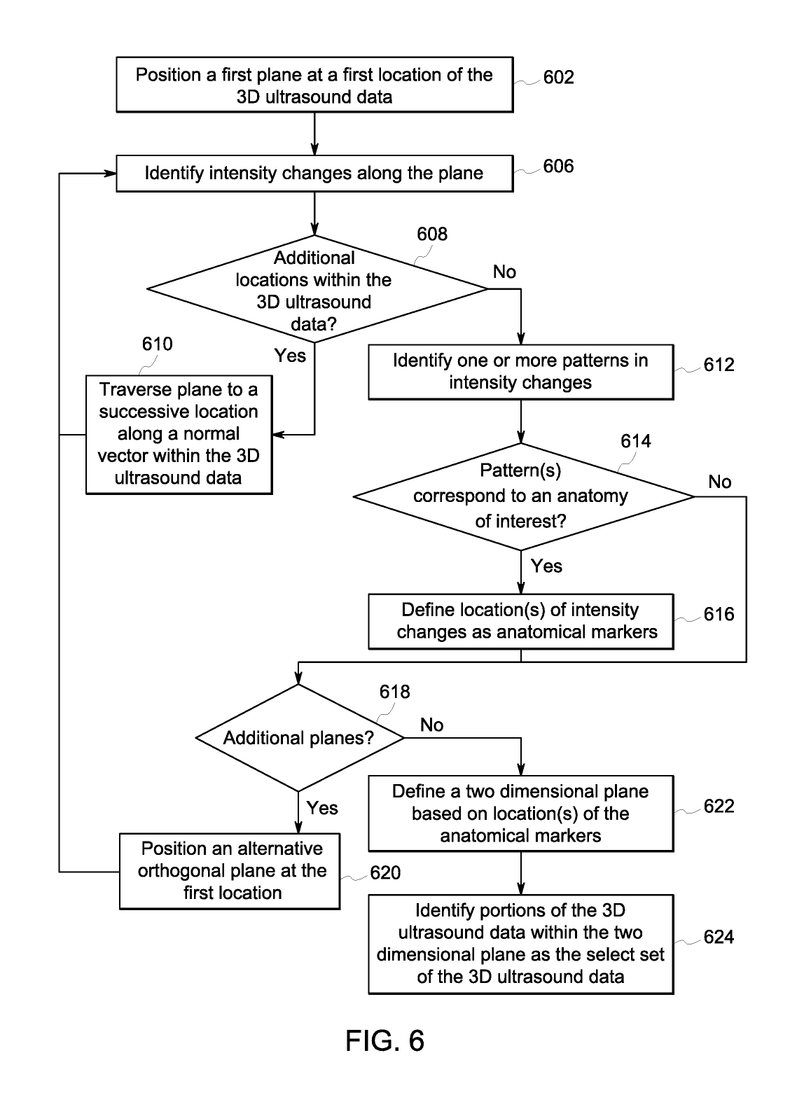

In connection with FIG. 6, the one or more patterns formed by the anatomical markers 552-558 may be verified by the controller circuit 136 to correspond to the anatomy of interest 550, and select a portion or set of the 3D ultrasound data that includes the anatomical markers 552-558.

FIG. 6 illustrate a flowchart of a method 600 for identifying a select set of the three dimensional ultrasound data that includes a plurality of anatomical markers, in accordance with various embodiments described herein. The method 600, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 600 may be used as one or more algorithms, such as a pattern recognition algorithm to direct the controller circuit 136 to perform one or more operations described herein. It may be noted, other methods may be used, in accordance with embodiments herein.

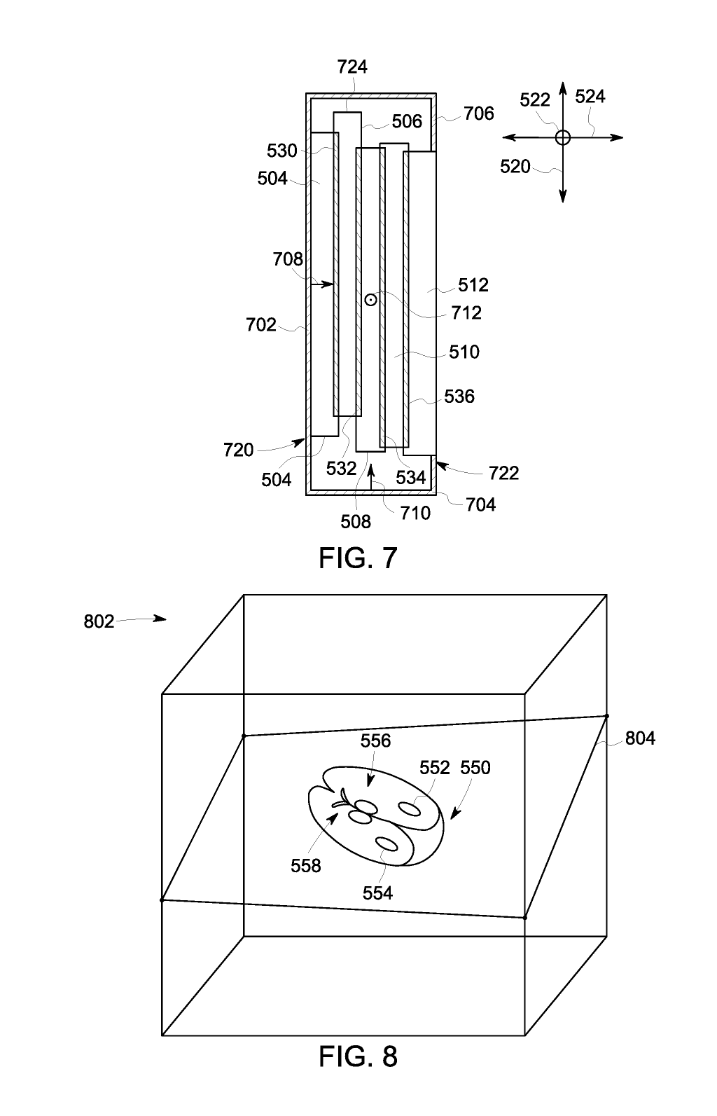

Beginning at 602, the controller circuit 136 may position a first plane at a first location of the 3D ultrasound data. FIG. 7 illustrates the frames 504-512 of the 3D ultrasound data shown in FIG. 5B with orthogonal planes 702-706. Each of the orthogonal planes 702-702 may be based on one of the axes 520-524 shown in FIGS. 5A-B and 7. For example, a first plane 702 may be based on (e.g., aligned with) the axis 520, representing a dimension of the 3D ultrasound data. The first location may correspond to an origin location of the 3D ultrasound data from where the first plane may traverse from. For example, a first location 720 is illustrated in FIG. 7. The first location 720 is positioned at an outer edge of the 3D ultrasound data, such as at a corner of the frame 504. It may be noted that in various other embodiments, the first location may be at other locations of the 3D ultrasound data. For example, in at least one embodiment the first location may be at an alternative corner of the frame 504 or the frame 512 with respect to the first location 720 shown in FIG. 7. The position of the first location 720 allows the first plane 702 to be exposed and/or interact with all or most (e.g., with respect to other positions) of the 3D ultrasound data when traversing and/or moving the first plane 702 from the first location 720 to an opposing location of the 3D ultrasound data, such as at 722.

At 606, the controller circuit 136 may identify intensity changes along the plane. For example, the controller circuit 136 may calculate a set of intensity gradients of the 3D ultrasound data at the first plane 702. The set of intensity gradients may be collections of calculated intensity gradient vectors or intensity gradient magnitudes corresponding to locations along the first plane 702 of the 3D ultrasound data. For example, the controller circuit 136 may calculate a derivative of the 3D ultrasound data corresponding to a change in intensity of the vector data along the first plane 702.

At 608, the controller circuit 136 may determine whether additional locations within the 3D ultrasound data need to be identified. If the controller circuit 136 determines that there are additional locations, at 610, the controller circuit 136 may traverse the plane to a successive location along a normal vector within the 3D ultrasound data. In operation, the controller circuit 136 may repeat the operation at 606 at different locations of the first plane 702 within the 3D ultrasound data until the controller circuit 136 identifies the intensity changes for all and/or over a predetermined threshold of the 3D ultrasound data (e.g., all of the frames 504-512). For example, the controller circuit 136 may move the first plane 702 in the direction of the normal vector 708 to a successive location. The successive location corresponding to a position within the 3D ultrasound data adjacent to the preceding location of the first plane 702 (e.g., the location of the first plane 702 at 606).

At 612, the controller circuit 136 may identify one or more patterns based on the intensity changes. Based on locations and magnitudes of the intensity changes (e.g., gradient values) identified within the 3D ultrasound data, the controller circuit 136 may identify shapes, contours, relative positions, and/or the like that form one or more patterns. For example, the controller circuit 136 may compare the intensity changes with an intensity change threshold. The intensity change threshold may correspond to a peak value, such as a gradient magnitude, that may indicate changes in adjacent pixel intensities that may represent an anatomical structure or marker within the volumetric ROI, such as one of the anatomical markers 552-558 shown in FIG. 5A. The controller circuit 136 may compare the intensity changes with the intensity change threshold to locate areas of interest that may correspond to anatomical markers. The controller circuit 136 may identify or define one or more patterns within the 3D ultrasound data that are formed by the locations of the areas of interest with respect to each other.

At 614, the controller circuit 136 may determine whether the pattern(s) corresponds to an anatomy of interest. For example, the controller circuit 136 may compare the one or more identified patterns at 612 with a plurality of patterns stored in the memory 140, 290. The plurality of patterns may each include a corresponding anatomy. In operation, the controller circuit 136 may calculate a differences between the one or more identified patterns and the plurality of patterns stored in the memory 140, 290. The controller circuit 136 may determine that the identified pattern corresponds to an anatomy of interest when the calculated difference between the identified pattern and one of the plurality patterns is below a predetermined error threshold. Optionally, the controller circuit 136 may select a portion of the identified pattern and/or subdivide the identified pattern, which may be compared by the controller circuit 136 with the plurality of patterns stored in the memory 140, 290.

Additionally or alternatively, the controller circuit 136 may execute a pattern recognition algorithm stored in the memory 140, 290. The pattern recognition algorithm may correspond to a machine learning algorithm based on a classifier (e.g., random forest classifier) that builds a model to label and/or assign each identified pattern by the controller circuit 136 into a corresponding anatomy of interest, background anatomy, and/or the like. The control circuit 136 when executing the pattern recognition algorithm may assign the identified pattern based on the various intensity changes and spatial positions of the intensity changes forming the pattern within the 3D ultrasound data.

If the controller circuit 136 determines that the identified pattern is of the anatomy of interest, at 616, the controller circuit 136 may define location(s) of the intensity changes as one or more anatomical markers. For example, the controller circuit 136 may assign and/or define the areas of interest forming the identified pattern having intensity changes above the intensity change threshold as the anatomical markers 552-558.

At 618, the controller circuit 136 determines whether additional planes are needed. If additional planes are needed, at 620, the controller circuit 136 may position an alternative orthogonal plane at the first location. For example, the controller circuit 136 may add an alternative orthogonal plane and return to 606 until the controller circuit 136 has identified intensity changes along three orthogonal planes.

In operation, the controller circuit 136 may traverse three orthogonal planes (e.g., the first plane 702, a plane 704, a plane 706) through the 3D ultrasound data. Each of the planes 702-706 may be orthogonal with respect to each other. The three orthogonal planes 702-706 may correspond to three dimensions of the 3D ultrasound data, such along the axes 520-524. For example, the plane 704 may be based on (e.g., aligned with) the axis 525, representing a dimension of the 3D ultrasound data. The controller circuit 136 may traverse the plane 704 within the 3D ultrasound data in the direction of a normal vector 710. In another example, the plane 706 may be based on (e.g., aligned with) the axis 522, representing a dimension of the 3D ultrasound data. The controller circuit 136 may traverse the plane 706 within the 3D ultrasound data in the direction of a normal vector 712. The controller circuit 136 may traverse the plane 704 and the plane 706 within the 3D ultrasound data to identify other locations corresponding to one or more of the anatomical markers 552-558 within the 3D ultrasound data, such as at 616.

At 622, the controller circuit 136 may define a 2D plane based on the anatomical markers. FIG. 8 illustrates a defined two dimensional plane 804 within 3D ultrasound data 802 based on one or more anatomical markers (e.g., the anatomical markers 552-558), in accordance with an embodiment. The 3D ultrasound data 802 may be based on the frames 504-512 shown in FIGS. 5A-B. For example, each of the anatomical markers 552-558 may have a location based on the three planes 702-706, which corresponds to a three dimensional coordinate within the 3D ultrasound data. The controller circuit 136 may define the 2D plane through the 3D ultrasound data to include or intercept the anatomical markers 552-558. Optionally, the controller circuit 136 may define alternate 2D planes through the 3D ultrasound data adjacent to and/or around the 2D plane 804.

At 624, the controller circuit 136 may identify portions of the 3D ultrasound data within the 2D plane as the select set of the 3D ultrasound data. For example, the controller circuit 136 may define the 3D ultrasound data included in the 2D plane 804 as a select set of the 3D ultrasound data. It may be noted that the controller circuit 136 may identify multiple sets of the 3D ultrasound data, each corresponding to different 2D planes.

Returning to FIG. 3, at 314, the controller circuit 136 generates a 2D ultrasound image based on the select set of the 3D ultrasound data. For example, the select set of the 3D ultrasound data may be stored on the memory 290. The scan converter circuit 292 (shown in FIG. 2) may access and obtain from the memory 290 the select set of the 3D ultrasound data, for example corresponding to the 2D plane 804. The scan converter circuit 292 may convert the 3D ultrasound data from vector data values to Cartesian coordinates to generate a 2D ultrasound image for the display 138. It may be noted that in various embodiments, the scan converter 292 may convert multiple 2D planes to generate a plurality of 2D ultrasound images for the display 138 corresponding to one or more anatomies of interest.

For example, the controller circuit 136 may identify a second select set of the 3D ultrasound data that includes a second plurality of anatomical markers corresponding to a second anatomy of interest. The controller circuit 136 may generate a second 2D ultrasound image based on the second select set of the 3D ultrasound data.

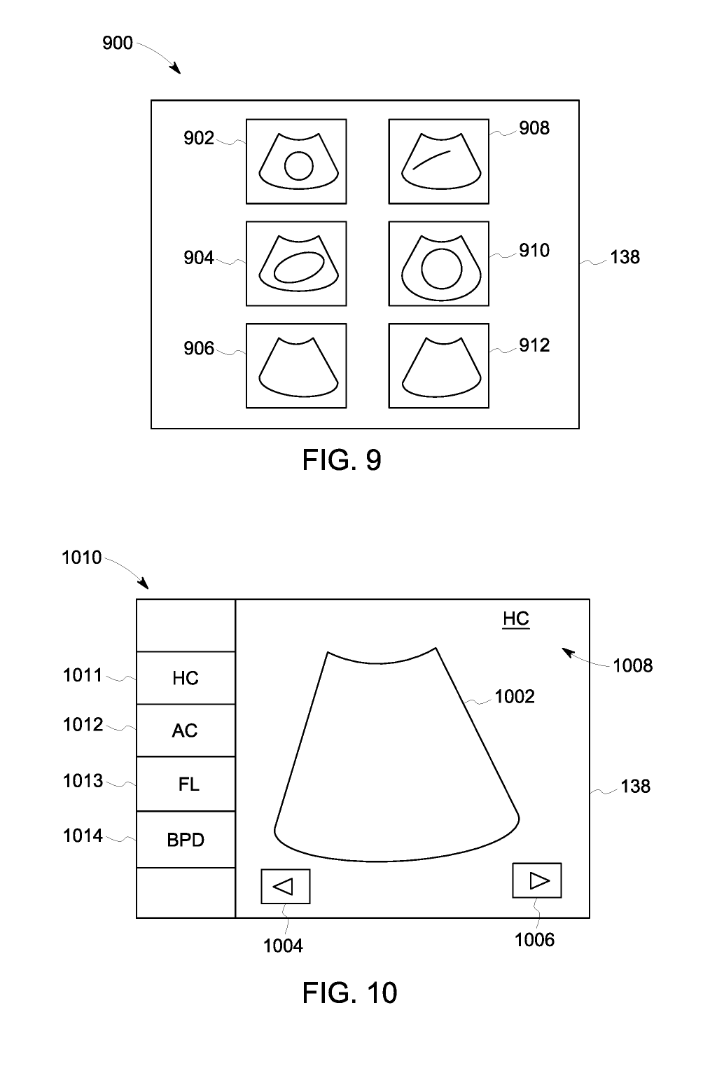

At 316, the controller circuit 136 displays the 2D ultrasound image on the display 138. FIG. 9 illustrates a graphical user interface 900 of a plurality of 2D ultrasound images 902-912, in accordance with an embodiment. The 2D ultrasound images 902-912 may be shown concurrently on the display 138. Each of the 2D ultrasound images 902-912 may correspond to different anatomies of interest. For example, each of the 2D ultrasound images 902-912 may be converted by the scan converter 292 from 3D ultrasound data corresponding to different 2D planes determined at 622. Optionally, the 2D ultrasound images 902-912 may include an interface component, allowing the user to modify and/or adjust the 2D ultrasound image 902-912. For example, the user may select one of the 2D ultrasound images 902-912 using the user interface 142 to change a view (e.g., zoom in, zoom out, expand) of the selected 2D ultrasound image, select an alternative 2D frame defining the selected 2D ultrasound image, perform diagnostic measurements on the selected 2D ultrasound image, and/or the like.

FIG. 10 illustrates a graphical user interface 1000 of a 2D ultrasound image 1002, in accordance with an embodiment. The 2D ultrasound image 1002 may be converted by the scan converter 292 from the 3D ultrasound data corresponding to the 2D plane 804. Additionally or alternatively, the 2D ultrasound image 1002 may correspond to one of the 2D ultrasound images 902-912 selected by the user using the user interface 142. The GUI 1000 may further display an identification code 1008 concurrently with the 2D ultrasound image 1002. The identification code 1008 may be a description of the anatomy of interest, a name of the patient, a date, and/or the like. The GUI 1000 may further include navigational interface components 1004-1006. The navigational interface components 1004-1006 may allow the user to toggle through and/or select an alternative 2D plane (e.g., adjacent to the 2D plane 804) corresponding to the anatomy of interest, select alternative 2D ultrasound images (e.g., the 2D ultrasound images 902-912) of different anatomies of interest, and/or the like. The GUI 1000 may further include a menu bar 1010 having one or more interface components 1011-1014. Each of the interface components 1011-1014 may correspond to a different anatomy of interest. For example, the user may view 2D ultrasound image of a different anatomies of interest by selecting a different interface component 1011-1014.

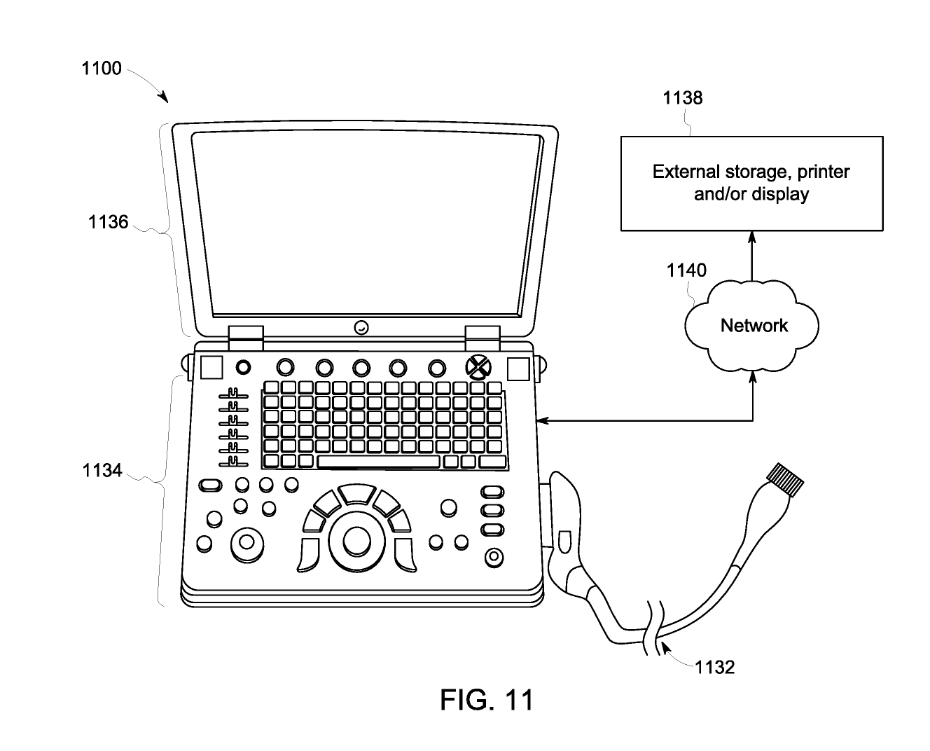

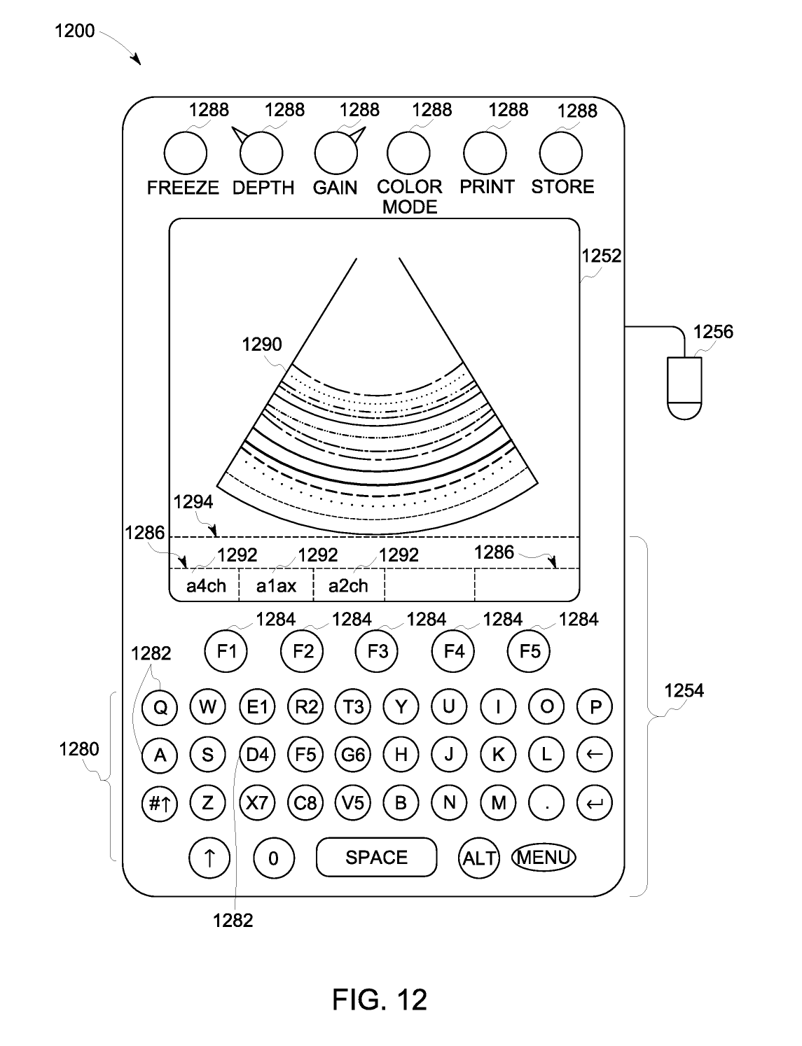

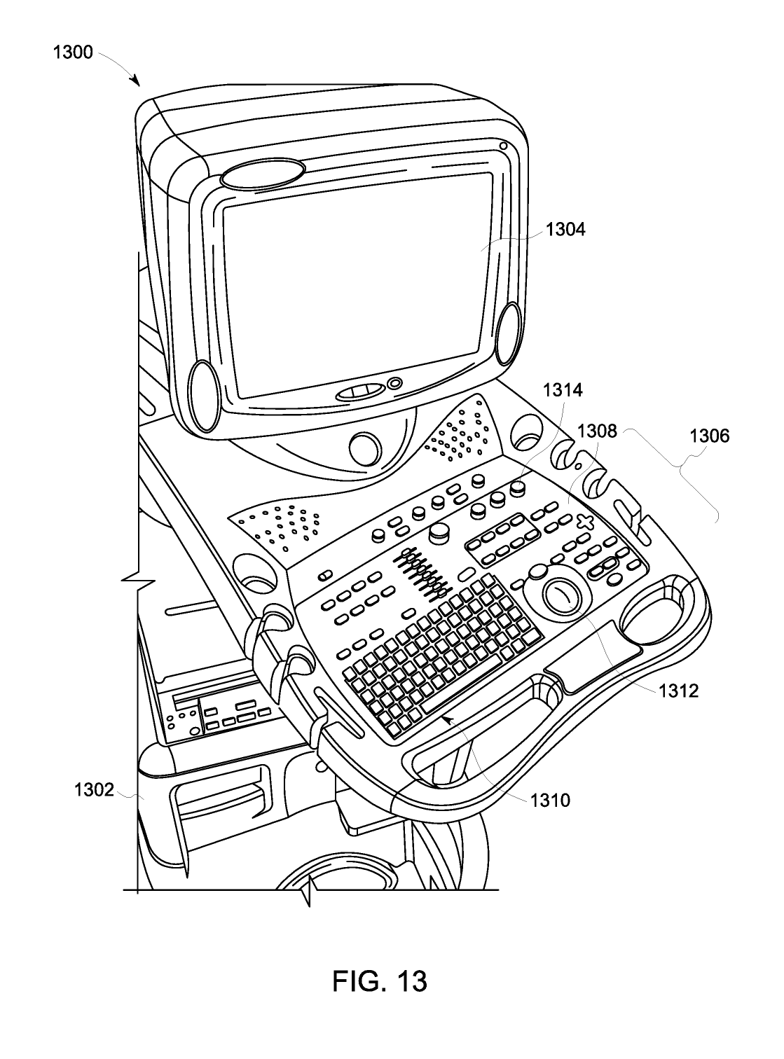

The ultrasound imaging system 100 of FIG. 1 may be embodied in a small-sized system, such as laptop computer or pocket-sized system as well as in a larger console-type system. FIGS. 11 and 12 illustrate small-sized systems, while FIG. 13 illustrates a larger system.

FIG. 11 illustrates a 3D-capable miniaturized ultrasound system 1130 having a probe 1132 that may be configured to acquire 3D ultrasonic data or multi-plane ultrasonic data. For example, the probe 1132 may have a 2D array of elements as discussed previously with respect to the probe. A user interface 1134 (that may also include an integrated display 1136) is provided to receive commands from an operator. As used herein, “miniaturized” means that the ultrasound system 1130 is a handheld or hand-carried device or is configured to be carried in a person's hand, pocket, briefcase-sized case, or backpack. For example, the ultrasound system 1130 may be a hand-carried device having a size of a typical laptop computer. The ultrasound system 1130 is easily portable by the operator. The integrated display 1136 (e.g., an internal display) is configured to display, for example, one or more medical images.

The ultrasonic data may be sent to an external device 1138 via a wired or wireless network 1140 (or direct connection, for example, via a serial or parallel cable or USB port). In some embodiments, the external device 1138 may be a computer or a workstation having a display. Alternatively, the external device 1138 may be a separate external display or a printer capable of receiving image data from the hand carried ultrasound system 1130 and of displaying or printing images that may have greater resolution than the integrated display 1136.

FIG. 12 illustrates a hand carried or pocket-sized ultrasound imaging system 1200 wherein the display 1252 and user interface 1254 form a single unit. By way of example, the pocket-sized ultrasound imaging system 1200 may be a pocket-sized or hand-sized ultrasound system approximately 2 inches wide, approximately 4 inches in length, and approximately 0.5 inches in depth and weighs less than 3 ounces. The pocket-sized ultrasound imaging system 1200 generally includes the display 1252, user interface 1254, which may or may not include a keyboard-type interface and an input/output (I/O) port for connection to a scanning device, for example, an ultrasound probe 1256. The display 1252 may be, for example, a 320×320 pixel color LCD display (on which a medical image 1290 may be displayed). A typewriter-like keyboard 1280 of buttons 1282 may optionally be included in the user interface 1254.

Multi-function controls 1284 may each be assigned functions in accordance with the mode of system operation (e.g., displaying different views). Therefore, each of the multi-function controls 1284 may be configured to provide a plurality of different actions. One or more interface components, such as label display areas 1286 associated with the multi-function controls 1284 may be included as necessary on the display 1252. The system 1200 may also have additional keys and/or controls 1288 for special purpose functions, which may include, but are not limited to “freeze,” “depth control,” “gain control,” “color-mode,” “print,” and “store.”

One or more of the label display areas 1286 may include labels 1292 to indicate the view being displayed or allow a user to select a different view of the imaged object to display. The selection of different views also may be provided through the associated multi-function control 1284. The display 1252 may also have one or more interface components corresponding to a textual display area 1294 for displaying information relating to the displayed image view (e.g., a label associated with the displayed image).

It may be noted that the various embodiments may be implemented in connection with miniaturized or small-sized ultrasound systems having different dimensions, weights, and power consumption. For example, the pocket-sized ultrasound imaging system 1200 and the miniaturized ultrasound system 1130 may provide the same scanning and processing functionality as the system 100.

FIG. 13 illustrates an ultrasound imaging system 1300 provided on a movable base 1302. The portable ultrasound imaging system 1300 may also be referred to as a cart-based system. A display 1304 and user interface 1306 are provided and it should be understood that the display 1304 may be separate or separable from the user interface 1306. The user interface 1306 may optionally be a touchscreen, allowing the operator to select options by touching displayed graphics, icons, and the like.

The user interface 1306 also includes control buttons 1308 that may be used to control the portable ultrasound imaging system 1300 as desired or needed, and/or as typically provided. The user interface 1306 provides multiple interface options that the user may physically manipulate to interact with ultrasound data and other data that may be displayed, as well as to input information and set and change scanning parameters and viewing angles, and/or the like. For example, a keyboard 1310, trackball 1312 and/or multi-function controls 1314 may be provided.

It may be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. A method for generating an ultrasound image, the method comprising:
acquiring three dimensional (3D) ultrasound data of a volumetric region of interest (ROI) from an ultrasound probe;
identifying a select set of the 3D ultrasound data that includes a plurality of anatomical markers, the one or more anatomical markers corresponding to an anatomy of interest within the volumetric ROI;
generating a two dimensional (2D) ultrasound image based on the select set of the 3D ultrasound data; and
displaying the 2D ultrasound image on a display.

2. The method of claim 1, wherein the display does not display an ultrasound image when acquiring the 3D ultrasound data.

3. The method of claim 1, further comprising:
identifying a second select set of the 3D ultrasound data that include a second plurality of anatomical markers corresponding to a second anatomy of interest, and
generating a second 2D ultrasound image based on the second select set of the 3D ultrasound data.

4. The method of claim 2, further comprising displaying the 2D ultrasound image and the second 2D ultrasound image concurrently on the display.

5. The method of claim 1, further comprising:
traversing a first plane within the 3D ultrasound data; and
identifying a location of at least one of the plurality of anatomical markers within the 3D ultrasound data with respect to the first plane.

6. The method of claim 5, further comprising traversing a second plane and a third plane within the 3D ultrasound data to identify other locations corresponding to one or more anatomical markers within the 3D ultrasound data, wherein each of the first plane, the second plane, and the third plane are orthogonal with respect to each other.

7. The method of claim 5, wherein the select set of the 3D ultrasound data is identified based on the location.

8. The method of claim 1, receiving the anatomy of interest from a user interface; and
adjusting acquisition settings of the ultrasound probe based on the anatomy of interest.

9. The method of claim 1, further comprising receiving a completion signal from a user interface when the acquisition of the 3D ultrasound data is complete, wherein the select set of the 3D ultrasound data is identified when the completion signal is received.

10. The method of claim 1, wherein the 3D ultrasound data includes vector data.

11. An ultrasound imaging system comprising:
an ultrasound probe configured to acquire three dimensional (3D) ultrasound data of a volumetric region of interest (ROI);
a display;
a memory configured to store programmed instructions; and
one or more processors configured to execute the programmed instructions stored in the memory, wherein the one or more processors when executing the programmed instructions perform the following operations:
collect the 3D ultrasound data from the ultrasound probe;
identify a select set of the 3D ultrasound data that includes a plurality of anatomical markers, the one or more anatomical markers corresponding to an anatomy of interest within the volumetric ROI;
generate a two dimensional (2D) ultrasound image based on the select set of the 3D ultrasound data; and
displaying the 2D ultrasound image on the display.

12. The ultrasound imaging system of claim 11, wherein an ultrasound image is not displayed on the display when the one or more processors collect the 3D ultrasound data

13. The ultrasound imaging system of claim 11, wherein the one or more processors further:
identify a second select set of the 3D ultrasound data that include a second plurality of anatomical markers corresponding to a second anatomy of interest, and
generate a second 2D ultrasound image based on the second select set of the 3D ultrasound data.

14. The ultrasound imaging system of claim 13, wherein the one or more processors further display the 2D ultrasound image and the second 2D ultrasound image concurrently on the display.

15. The ultrasound imaging system of claim 11, wherein the one or more processors further traverse a first plane within the 3D ultrasound data, and identify a location of at least one of the plurality of anatomical markers within the 3D ultrasound data with respect to the first plane.

16. The ultrasound imaging system of claim 15, wherein the select set of the 3D ultrasound data is identified based on the location.

17. The ultrasound imaging system of claim 11, further comprising a user interface configured to transmit a completion signal corresponding to completion of the 3D ultrasound data, wherein the select set of the 3D ultrasound data is identified by the one or more processors when the completion signal is received.

18. The ultrasound imaging system of claim 11, wherein the 3D ultrasound data includes vector data.

19. A tangible and non-transitory computer readable medium comprising one or more computer software modules configured to direct one or more processors to:
acquire three dimensional (3D) ultrasound data of a volumetric region of interest (ROI) from an ultrasound probe;
identify a select set of the 3D ultrasound data that includes a plurality of anatomical markers, the one or more anatomical markers corresponding to an anatomy of interest within the volumetric ROI;
generate a two dimensional (2D) ultrasound image based on the select set of the 3D ultrasound data; and
display the 2D ultrasound image on a display.

20. The tangible and non-transitory computer readable medium of claim 19, wherein the one or more processors are further directed to
identify a second select set of the 3D ultrasound data that include a second plurality of anatomical markers corresponding to a second anatomy of interest, and
generate a second 2D ultrasound image based on the second select set of the 3D ultrasound data.
